(19) United States
(12) Patent Application Publication
(10) Pub. No.: US 2007/0232685 A1
(43) Pub. Date: Oct. 4, 2007

(54) CRYSTALLINE FORMS OF N-(BENZO[B]THIEN-3-YLMETHYL)-SULFAMIDE

(76) Inventors: Nagy Fawzy, Piscataway, NJ (US); Michael H. Parker, Chalfont, PA (US); Allen B. Reitz, Lansdale, PA (US); Bruce E. Maryanoff, Forest Grove, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/677,717

(22) Filed: Feb. 22, 2007

Related U.S. Application Data
(60) Provisional application No. 60/775,724, filed on Feb. 22, 2006.

(51) Int. Cl. A61K 31/381; C07D 333/56

(52) U.S. Cl. 514/443; 549/58

(57) ABSTRACT

The present invention is directed to anhydrous and solvated, crystalline forms of N-(benzo[b]thien-3-ylmethyl)-sulfamide, pharmaceutical compositions containing said crystalline forms and their use in the treatment epilepsy and related disorders.

### CROSS REFERENCE TO RELATED APPLICATIONS

The application claims the benefit of U.S. Provisional Application 60/775,724, filed on Feb. 22, 2006, which is incorporated by reference herein in it's entirety.

### FIELD OF THE INVENTION

The present invention is directed to novel anhydrous and solvate crystalline forms of N-(benzo[b]thien-3-ylmethyl)-sulfamide, pharmaceutical compositions containing said crystalline forms and their use in the treatment epilepsy and related disorders.

### BACKGROUND OF THE INVENTION

Epilepsy describes a condition in which a person has recurrent seizures due to a chronic, underlying process. Epilepsy refers to a clinical phenomenon rather than a single disease entity, since there are many forms and causes of epilepsy. Using a definition of epilepsy as two or more unprovoked seizures, the incidence of epilepsy is estimated at approximately 0.3 to 0.5 percent in different populations throughout the world, with the prevalence of epilepsy estimated at 5 to 10 people per 1000.

An essential step in the evaluation and management of a patient with a seizure is to determine the type of seizure that has occurred. The main characteristic that distinguishes the different categories of seizures is whether the seizure activity is partial (synonymous with focal) or generalized.

Partial seizures are those in which the seizure activity is restricted to discrete areas of the cerebral cortex. If consciousness is fully preserved during the seizure, the clinical manifestations are considered relatively simple and the seizure is termed a simple-partial seizure. If consciousness is impaired, the seizure is termed a complex-partial seizure. An important additional subgroup comprises those seizures that begin as partial seizures and then spread diffusely throughout the cortex, which are known as partial seizures with secondary generalization.

Generalized seizures involve diffuse regions of the brain simultaneously in a bilaterally symmetric fashion. Absence or petit mal seizures are characterized by sudden, brief lapses of consciousness without loss of postural control. Atypical absence seizures typically include a longer duration in the lapse of consciousness, less abrupt onset and cessation, and more obvious motor signs that may include focal or lateralizing features. Generalized Tonic-clonic or grand mal seizures, the main type of generalized seizures, are characterized by abrupt onset, without warning. The initial phase of the seizure is usually tonic contraction of muscles, impaired respiration, a marked enhancement of sympathetic tone leading to increased heart rate, blood pressure, and pupillary size. After 10-20 s, the tonic phase of the seizure typically evolves into the clonic phase, produced by the superimposition of periods of muscle relaxation on the tonic muscle contraction. The periods of relaxation progressively increase until the end of the ictal phase, which usually lasts no more than 1 min. The postictal phase is characterized by unresponsiveness, muscular flaccidity, and excessive salivation that can cause stridorous breathing and partial airway obstruction. Atonic seizures are characterized by sudden loss of postural muscle tone lasting 1-2 s. Consciousness is briefly impaired, but there is usually no postictal confusion. Myoclonic seizures are characterized by a sudden and brief muscle contraction that may involve one part of the body or the entire body.

Carbonic anhydrase inhibitors (CAIs) have been widely used in medicine, mainly as antiglaucoma and antisecretory drugs or diuretic agents, and are valuable compounds. However, systemic antiglaucoma agents (such as acetazolamide) possess potentially unwanted side-effects including paresthesias, nephrolithiasis and weight loss. Topiramate is a well known anticonvulsant drug that possesses single digit micromolar carbonic anhydrase inhibition, which is suspected as the cause of paresthesias noted by some patients taking topiramate.

### SUMMARY OF THE INVENTION

The present invention is directed to crystalline forms of the compound of formula (I)

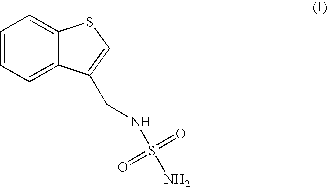

also known as, N-(benzo[b]thien-3-ylmethyl)-sulfamide. Such crystalline forms include those crystalline forms comprising the X-ray diffraction patterns exemplified in FIGS. 2-5 or the diffraction patterns with the peaks listed in Tables 4-11.

In an embodiment, the present invention is directed to novel anhydrous crystalline forms of the compound of formula (I). In another embodiment, the present invention is directed to a novel solvated crystalline form of the compound of formula (I), more specifically a novel 1-4-dioxane solvated crystalline form of the compound of formula (I).

In another embodiment, the present invention is directed to a novel crystalline form of the compound of formula (I)

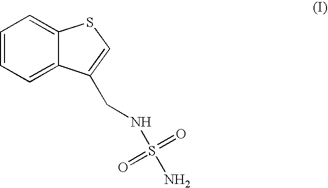

wherein the crystalline form is characterized by a DSC temperature of onset selected from the group consisting of about 95° C., about 98° C. and about 101° C.

Illustrative of the invention is a pharmaceutical composition comprising a pharmaceutically acceptable carrier and any of the crystalline forms described above. An illustration of the invention is a pharmaceutical composition made by mixing any of the crystalline forms described above and a pharmaceutically acceptable carrier. Illustrating the invention is a process for making a pharmaceutical composition comprising mixing any of the crystalline forms described above and a pharmaceutically acceptable carrier.

Exemplifying the invention is a method of treating epilepsy and related disorders comprising administering to a subject in need thereof a therapeutically effective amount of any of the crystalline forms or pharmaceutical compositions described above.

Another example of the invention is the use of any of the crystalline forms described herein in the preparation of a medicament for treating epilepsy or a related disorder, in a subject in need thereof.

### BRIEF DESCRIPTION OF THE FIGURES

FIG. 1 illustrates the measured X-ray diffraction pattern for a representative sample of novel anhydrous crystalline form A-I.

FIG. 2 illustrates the measured X-ray diffraction pattern for a representative sample of novel anhydrous crystalline form A-II.

FIG. 3 illustrates the measured X-ray diffraction pattern for a representative sample of novel anhydrous crystalline form A-III.

FIG. 4 illustrates the measured X-ray diffraction pattern for a representative sample of novel anhydrous crystalline form A-IV.

FIG. 5: illustrates the measured X-ray diffraction pattern for a representative sample of novel solvated crystalline form S-I.

### DETAILED DESCRIPTION OF THE INVENTION

The present invention is directed to novel crystalline forms of the compound of formula (I)

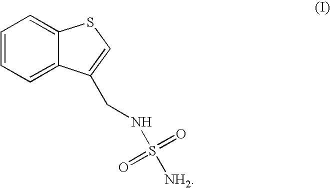

More specifically, the present invention is directed to four novel anhydrous crystalline forms of the compound of formula (I), hereinafter referred to as Form A-I, A-II, A-III and A-IV; and one novel solvate form of the compound of formula (I), a 1-4-dioxane solvated crystalline form of the compound of formula (I), hereinafter referred to as Form S-I.

The novel crystalline forms of the compound of formula (I) were prepared as follows. The compound of formula (I) as Form A-I (20 mg) (prepared as in Example 1 below) was placed into a 4 mL vial. The selected solvents were then added to the vials to form a saturated solution according to the table below. The resulting mixture was heated at 40° C. on a hot plate, then stirred, vortexed and sonicated for 2 min. The vials were removed form the hot plate and held at room temperature to cool down, and then filtered using 0.45 μm FTPE filter cartridge and supernatants were used for crystallization/precipitation study.

The vials were then capped, but not sealed and kept at room temperature in a hood to facilitate slow evaporation of the solvent to yield a residue. (For solvent choices of 1,4-dioxane, butanol and water, after removing from the vial from the hot plate, the solvent was instead removed under vacuum.) An XRD pattern was then measured for each residue to determine crystalline form.

Table 1, below lists the solvents used in the preparation of the novel crystalline forms of the compound of formula (I) and the resulting form, as determined by XRD.

TABLE 1

| | Solvent | Resulting | |
|---|---|---|---|
| Solvent | Added (mL) | Mixture | Crystalline Form |
| Methanol | 1 | Clear Solution | A-II |
| Ethanol | 3 | Suspension | A-I |
| Acetonitrile | 3 | Suspension | mixture A-I and A-III |
| Acetone | 3 | Suspension | A-III |
| Methyl Acetate | 3 | Suspension | A-I |
| Ethyl Acetate | 3 | Suspension | mixture A-I and A-III |
| Isopropyl Alcohol | 3 | Suspension | mixture A-I and A-IV |
| Nitromethane | 3 | Suspension | mixture A-I and A-II |
| 1,4-Dioxane | 3 | Suspension | S-I (sticky residue) |
| Toluene | 4 | Suspension | A-I |
| Hexane | 4 | Suspension | A-I |
| Butanol | 3 | Suspension | mixture A-I and A-III |
| Dichloromethane | 3 | Suspension | A-III |
| Water | 4 | Suspension | A-I |

The novel crystalline forms of the compound of formula (I) may be characterized by their Powder X-Ray Diffraction (XRD) patterns. XRD patterns were measured for the novel crystalline forms of the compound of formula (I) using a D8 X-ray Diffractometer from Bruker, which consisted of a KRISTALLOFLEX 760 X-ray generator with Cu anode tube, running at 45 KV 40 mA. Samples were placed on a zero background XRD sample holder and scanned from 3° to 40° 2θ at a scan rate of 0.0167° 2θ/second.

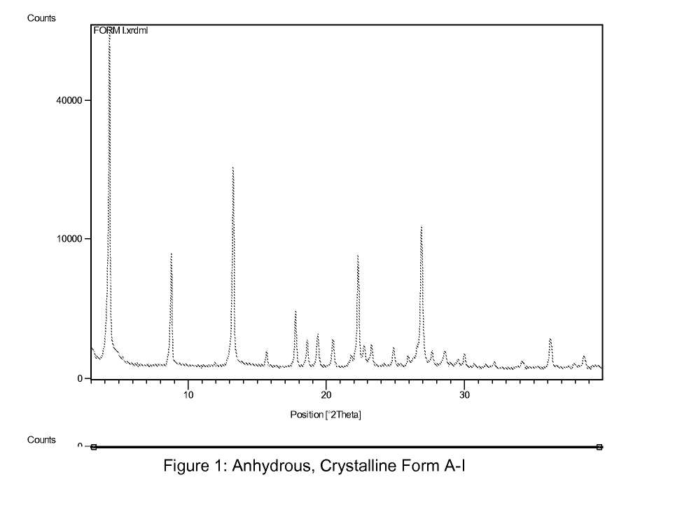

Crystalline, anhydrous Form A-I may be characterized by its X-ray diffraction pattern, a representative example of which is shown in FIG. 1. Crystalline, anhydrous Form A-I may alternatively be characterized by its X-ray diffraction pattern, comprising the representative peaks as listed in Table 2, below.

TABLE 2
Form A-I

| Position | d-spacing | Relative |
|---|---|---|
| [°2Theta] | [Å] | Intensity [%] |
| 4.47 | 19.80 | 100 |
| 8.93 | 9.90 | 12.29 |
| 13.42 | 6.60 | 35.72 |
| 17.93 | 4.95 | 3.47 |
| 18.75 | 4.73 | 1.04 |
| 19.52 | 4.55 | 1.45 |
| 20.66 | 4.30 | 1.08 |
| 22.46 | 4.00 | 12.08 |
| 22.90 | 3.88 | 0.78 |
| 23.43 | 3.80 | 0.83 |
| 27.03 | 3.30 | 18.18 |
| 36.32 | 2.47 | 1.2 |
| 36.43 | 2.40 | 0.75 |

Preferably, crystalline, anhydrous Form A-I may be characterized by its XRD pattern which comprises the major peaks having a relative intensity greater than or equal to about 1%, as listed in Table 3 below.

TABLE 3
Form A-I

| Position | d-spacing | Relative |
|---|---|---|
| [°2Theta] | [Å] | Intensity [%] |
| 4.47 | 19.80 | 100 |
| 8.93 | 9.90 | 12.29 |
| 13.42 | 6.60 | 35.72 |
| 17.93 | 4.95 | 3.47 |
| 18.75 | 4.73 | 1.04 |
| 19.52 | 4.55 | 1.45 |
| 20.66 | 4.30 | 1.08 |
| 22.46 | 4.00 | 12.08 |
| 27.03 | 3.30 | 18.18 |
| 36.32 | 2.47 | 1.2 |

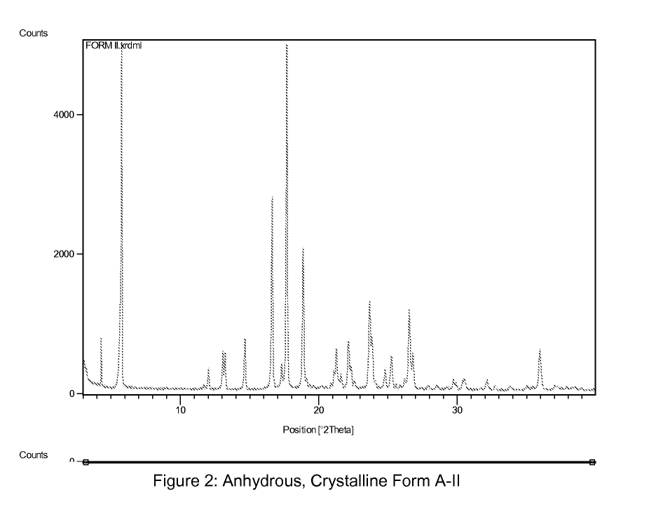

Crystalline, anhydrous Form A-II may be characterized by its X-ray diffraction pattern, a representative example of which is shown in FIG. 2. Crystalline, anhydrous Form A-II may alternatively be characterized by its X-ray diffraction pattern, comprising the representative peaks as listed in Table 4, below.

TABLE 4
Form A-II

| Position | d-spacing | Relative |
|---|---|---|
| [°2Theta] | [Å] | Intensity [%] |
| 4.44 | 19.91 | 14.21 |
| 5.92 | 14.94 | 100 |
| 13.21 | 6.70 | 10.7 |
| 13.38 | 6.62 | 10.29 |
| 14.80 | 5.99 | 14.5 |
| 16.76 | 5.29 | 54.91 |
| 17.81 | 4.98 | 99.33 |
| 19.00 | 4.67 | 40.12 |
| 21.43 | 4.15 | 11.61 |
| 22.29 | 3.99 | 13.84 |
| 23.82 | 3.74 | 25.39 |
| 24.01 | 3.71 | 14.8 |
| 26.67 | 3.34 | 22.69 |
| 26.95 | 3.31 | 10.39 |
| 36.10 | 2.49 | 11.51 |

Preferably, crystalline, anhydrous Form A-II is characterized by its XRD pattern which comprises peaks having a relative intensity greater than or equal to about 14%, as listed in Table 5 below.

TABLE 5
Form A-II

| Position | d-spacing | Relative |
|---|---|---|
| [°2Theta] | [Å] | Intensity [%] |
| 4.44 | 19.91 | 14.21 |
| 5.92 | 14.94 | 100 |
| 14.80 | 5.99 | 14.5 |
| 16.76 | 5.29 | 54.91 |
| 17.81 | 4.98 | 99.33 |
| 19.00 | 4.67 | 40.12 |
| 21.43 | 4.15 | 11.61 |
| 22.29 | 3.99 | 13.84 |
| 23.82 | 3.74 | 25.39 |
| 24.01 | 3.71 | 14.8 |
| 26.67 | 3.34 | 22.69 |

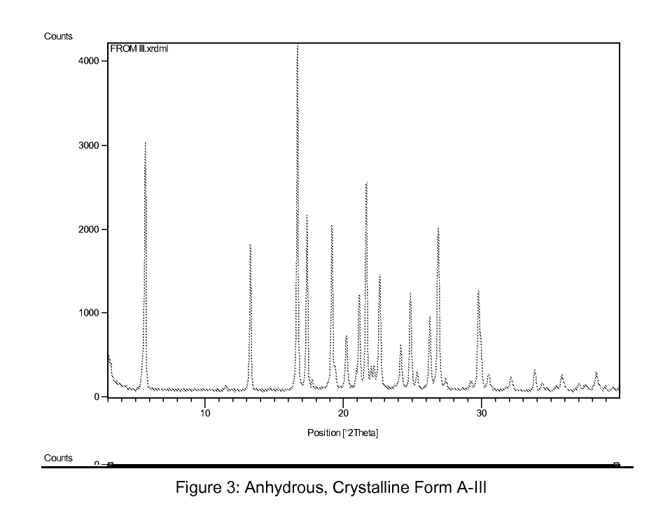

Crystalline, anhydrous Form A-III may be characterized by its X-ray diffraction pattern, a representative example of which is shown in FIG. 3. Crystalline, anhydrous Form A-III may alternatively be characterized by its X-ray diffraction pattern, comprising the representative peaks as listed in Table 6, below.

TABLE 6
Form A-III

| Position | d-spacing | Relative |
|---|---|---|
| [°2Theta] | [Å] | Intensity [%] |
| 5.84 | 15.14 | 71.34 |
| 13.42 | 6.60 | 42.01 |
| 16.81 | 5.27 | 100 |
| 17.50 | 5.07 | 50.58 |
| 19.30 | 4.60 | 47.27 |
| 20.37 | 4.36 | 15.62 |
| 21.30 | 4.17 | 27.74 |
| 21.81 | 4.07 | 60.53 |
| 22.78 | 3.90 | 33.42 |
| 24.29 | 3.66 | 13.37 |
| 24.99 | 3.56 | 27.14 |
| 26.40 | 3.38 | 21.56 |
| 26.98 | 3.30 | 46.5 |
| 29.89 | 2.99 | 27.38 |

Preferably, crystalline, anhydrous Form A-III is characterized by its XRD pattern which comprises peaks having a relative intensity greater than or equal to about 25%, as listed in Table 7 below.

TABLE 7
Form A-III

| Position | d-spacing | Relative |
|---|---|---|
| [°2Theta] | [Å] | Intensity [%] |
| 5.84 | 15.14 | 71.34 |
| 13.42 | 6.60 | 42.01 |
| 16.81 | 5.27 | 100 |
| 17.50 | 5.07 | 50.58 |
| 19.30 | 4.60 | 47.27 |
| 21.30 | 4.17 | 27.74 |
| 21.81 | 4.07 | 60.53 |
| 22.78 | 3.90 | 33.42 |
| 24.99 | 3.56 | 27.14 |
| 26.98 | 3.30 | 46.5 |
| 29.89 | 2.99 | 27.38 |

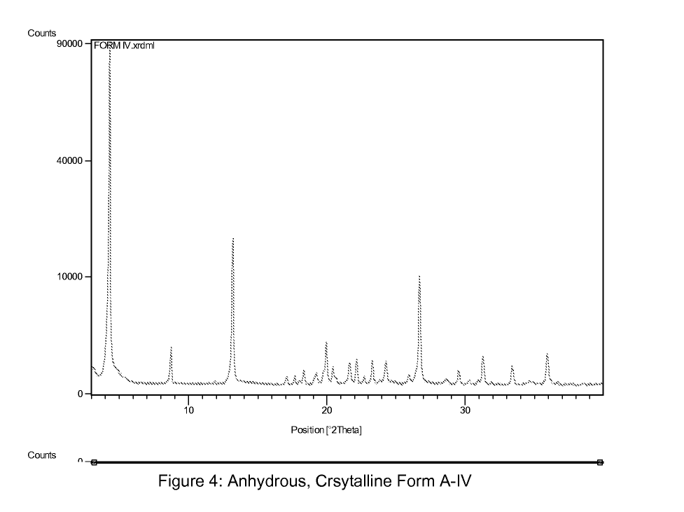

Crystalline, anhydrous Form A-IV may be characterized by its X-ray diffraction pattern, a representative example of which is shown in FIG. 4. Crystalline, anhydrous Form A-IV may alternatively be characterized by its X-ray diffraction pattern, comprising the representative peaks as listed in Table 6, below.

TABLE 8
Form A-IV

| Position | d-spacing | Relative |
|---|---|---|
| [°2Theta] | [Å] | Intensity [%] |
| 4.46 | 19.80 | 100 |
| 8.90 | 9.94 | 1.61 |
| 13.35 | 6.63 | 18.6 |
| 20.12 | 4.41 | 2.11 |
| 20.61 | 4.31 | 0.51 |
| 21.81 | 4.08 | 0.69 |
| 22.32 | 3.98 | 0.87 |
| 23.44 | 3.80 | 0.80 |
| 24.42 | 3.65 | 0.68 |
| 26.85 | 3.32 | 11.11 |
| 31.43 | 2.85 | 1.1 |
| 33.52 | 2.67 | 0.53 |
| 36.03 | 2.49 | 1.12 |

Preferably, crystalline, anhydrous Form A-IV is characterized by its XRD pattern which comprises peaks having a relative intensity greater than or equal to about 1 %, as listed in Table 9 below.

TABLE 9
Form A-IV

| Position | d-spacing | Relative |
|---|---|---|
| [°2Theta] | [Å] | Intensity [%] |
| 4.46 | 19.80 | 100 |
| 8.90 | 9.94 | 1.61 |
| 13.35 | 6.63 | 18.6 |
| 20.12 | 4.41 | 2.11 |
| 26.85 | 3.32 | 11.11 |
| 31.43 | 2.85 | 1.1 |
| 36.03 | 2.49 | 1.12 |

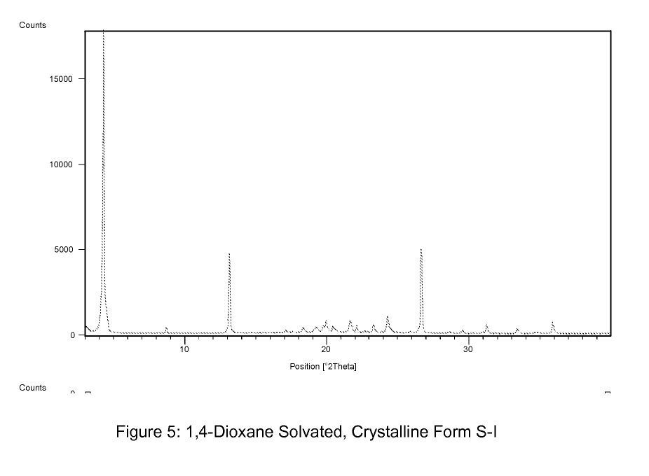

The crystalline, 1,4-dioxane solvated Form S-I may be characterized by its X-ray diffraction pattern, a representative example of which is shown in FIG. 5. Crystalline, solvated Form S-I may alternatively be characterized by its X-ray diffraction pattern, comprising the representative peaks as listed in Table 6, below.

TABLE 10
Form S-I

| Position | d-spacing | Relative |
|---|---|---|
| [°2Theta] | [Å] | Intensity [%] |
| 4.44 | 19.89 | 100 |
| 8.86 | 9.98 | 1.67 |
| 13.30 | 6.66 | 26.6 |
| 18.48 | 4.80 | 1.73 |
| 19.38 | 4.58 | 1.99 |
| 19.95 | 4.45 | 2.31 |
| 20.13 | 4.41 | 4.00 |
| 20.60 | 4.31 | 2.34 |
| 21.80 | 4.08 | 4.46 |
| 22.28 | 3.99 | 2.46 |
| 23.43 | 3.80 | 2.71 |
| 24.42 | 3.65 | 5.26 |
| 26.80 | 3.33 | 27.75 |
| 31.38 | 2.85 | 3.07 |
| 33.51 | 2.67 | 1.59 |
| 36.01 | 2.49 | 3.73 |

Preferably, crystalline, 1,4-dioxane solvated Form S-I is characterized by its XRD pattern which comprises peaks having a relative intensity greater than or equal to about 5%, as listed in Table 11 below.

TABLE 11
Form S-I

| Position | d-spacing | Relative |
|---|---|---|
| [°2Theta] | [Å] | Intensity [%] |
| 4.44 | 19.89 | 100 |
| 13.30 | 6.66 | 26.6 |
| 24.42 | 3.65 | 5.26 |
| 26.80 | 3.33 | 27.75 |

In an embodiment, the crystalline form of the compound of formula (I) is characterized by the peak positions (in °2θ) in the XRD spectra, wherein the peaks exhibit a relative intensity greater than about 1%, preferably, with a relative intensity greater than about 5%, more preferably, with a relative intensity greater than about 10%, more preferably, with a relative intensity greater than about 25%.

The crystalline forms of the compound of formula (I) were measured using a Diamond Differential Scanning Calorimeter (DSC). The samples (˜2-3 mg) were sealed in a 50 μL aluminum pan with a single 50 mm hole. The reference and sample pans were identical. The sample was scanned from 30° C. to 250° C. at 10°/min with a 40 mL/min nitrogen purge.

Table 12 below lists the temperature of onset and heat of fusion for the crystalline forms of the compound of formula (I), measured/calculated from the DSC measurements.

TABLE 12
DSC Data for Crystalline Forms

| | DSC Tₒₙₛₑₜ | Heat of Fusion |
|---|---|---|
| | (° C.) | (ΔH) (J/g) |
| Form A-I | 106.1 ± 1.0 | 109.1 |
| Form A-II | 94.9 ± 5.0^{a} | 98.2 |
| Form A-III | 98.1 ± 5.0^{a} | 73.4 |
| Form A-IV | 100.9 ± 1.0 | 105.0 |

^{a}For this sample the DSC peak broad

In an embodiment, the present invention is directed to a crystalline form of the compound of formula (I)

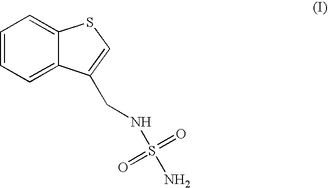

wherein the crystalline form is characterized by DSC temperature of onset selected from the group consisting of about 95° C., about 98° C. and about 101° C.

Representative samples of the novel crystalline forms of the compound of formula (I) were further tested on a PYRIS 1 Thermogravimetric Analyzer (TGA). The sample (˜2-3 mg) was heated at a scanning rate of 10° C./min between 30° C. and 250° C. Aluminum pans were used for all samples. Samples which exhibited no weight loss in the TGA scan were deemed to be anhydrous. If weight loss is observed in the TGA scan, the amount of weight lost was used to determine the percentage of the hydrate or solvate.

Crystalline forms A-I A-II, A-III and A-IV were determined to be anhydrous, wherein crystalline form S-I was determined to be a solvate, with the measured weight loss (Delta Y) was 8.71%.

As used herein, unless otherwise noted, the terms “epilepsy and related disorders” or “epilepsy or related disorder” shall mean any disorder in which a subject (preferably a human adult, child or infant) experiences one or more seizures and/or tremors. Suitable examples include, but are not limited to, epilepsy (including, but not limited to, localization-related epilepsies, generalized epilepsies, epilepsies with both generalized and local seizures, and the like), seizures as a complication of a disease or condition (such as seizures associated with encephalopathy, phenylketonuria, juvenile Gaucher's disease, Lundborg's progressive myoclonic epilepsy, stroke, head trauma, stress, hormonal changes, drug use or withdrawal, alcohol use or withdrawal, sleep deprivation, and the like), essential tremor, restless limb syndrome, and the like. Preferably, the disorder is selected from epilepsy (regardless of type, underlying cause or origin), essential tremor or restless limb syndrome, more preferably, the disorder is epilepsy (regardless of type, underlying cause or origin) or essential tremor.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

The present invention further comprises pharmaceutical compositions containing one or more of the novel crystalline forms of the compound of formula (I) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.1-1000 mg and may be given at a dosage of from about 0.01-150.0 mg/kg/day, preferably from about 0.1 to 100 mg/kg/day, more preferably from about 0.5-50 mg/kg/day, more preferably from about 1.0-25.0 mg/kg/day or any range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 1000 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating alcohol abuse and/or addiction described in the present invention may also be carried out using a pharmaceutical composition comprising any of the compounds as defined herein and a pharmaceutically acceptable carrier. The pharmaceutical composition may contain between about 0.1 mg and 1000 mg, preferably about 50 to 500 mg, of the compound, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

Compounds of this invention may be administered in any of the foregoing compositions and according to dosage regimens established in the art whenever treatment of alcohol abuse and/or addiction is required.

The daily dosage of the products may be varied over a wide range from 0.01 to 150 mg/kg per adult human per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.01 mg/kg to about 1500 mg/kg of body weight per day. Preferably, the range is from about 0.1 to about 100.0 mg/kg of body weight per day, more preferably, from about 0.5 mg/kg to about 50 mg/kg, more preferably, from about 1.0 to about 25.0 mg/kg of body weight per day. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

### EXAMPLE 1

### N-(benzo[b]thien-3-ylmethyl)-sulfamide

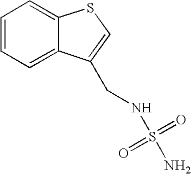

Thianaphthene-3-carboxaldehyde (1.62 g, 10.0 mmol) was dissolved in anhydrous ethanol (50 mL). Sulfamide (4.0 g, 42 mmol) was added and the mixture was heated to reflux for 16 hours. The mixture was cooled to room temperature. Sodium borohydride (0.416 g, 11.0 mmol) was added and the mixture was stirred at room temperature for three hours. The reaction was diluted with water (50 mL) and extracted with chloroform (3×75 mL). The extracts were concentrated and chromatographed (5% methanol in DCM) and the solvent evaporate to yield the title compound as a white solid.

¹H NMR (DMSO-d₆): δ 7.98 (1H, dd, J=6.5, 2.3 Hz), 7.92 (1H, dd, J=6.6, 2.4 Hz), 7.62 (1H, s), 7.36-7.45 (2H, m), 7.08 (1H, t, J=6.3 Hz), 6.72 (2H, s), 4.31 (2H, d, J=6.3 Hz).

### EXAMPLE 2

### Recrystallizaton of N-[(Benzo[b]thiophen-3-yl)methyl]sulfamide from Water

A 30 gal reactor was charged with crude N-[(benzo[b]thiophen-3-yl)methyl]sulfamide (470 g; 1.94 moles) followed by addition of water (25 L). The stirred mixture was heated to reflux and the heating was maintained until dissolution of the solid occurred. At this point the solution was hot filtered under pressure through an inline filter to a receiving vessel (20 gal) over a period of 30 minutes. The solution was then cooled to room temperature, over 2.5h. The resulting solid was collected by filtration and rinsed with water, then air-dried under vacuum overnight to yield the title compound as a white solid.

### EXAMPLE 3

### Recrystallizaton of N-[(Benzo[b]thiophen-3-yl)methyl]sulfamide from MTBE/Water

A 4 L Erlenmeyer flask was charged with crude N-[(Benzo[b]thiophen-3-yl)methyl]sulfamide (720 g; 2.97 moles) followed by addition of methyl tert-butyl ether (2.5 L) and water (80.0 mL, 4.44 mole) and the mixture was heated slowly to reflux. The resulting solution was hot filtered through a pad of Celite® into a 5 L four-necked reaction flask pre-warmed to 40° C. and equipped with an overhead stirrer, heating mantle, temperature control unit and vacuum adapter. The filter pad was washed with methyl tert-butyl ether (40 mL). After filtration the filtrate was allowed to cool slowly. When the temperature reached 60° C., the solution was seeded with a small amount of pure product, which induced crystallization of product shortly thereafter. Slow cooling was continued to room temperature and the mixture was maintained at room temperature overnight. The mixture was further cooled in an ice bath to 5° C. and the solid was collected by filtration, then air-dried to yield the title compound as a crystalline product

DSC m.p. 106.8° C.

Elemental analysis calculated for C₉H₁₀N₂O₂S₂:
Calculated: C, 44.61; H, 4.16; N, 11.56; O, 13.21; S; KF:26.47%
Measured: C: 44.43, H: 3.87, N: 11.57, S; KF 26.23%

### EXAMPLE 4

As a specific embodiment of an oral composition, 100 mg of novel crystalline Form A-I, prepared as in Example 1, is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. An anhydrous crystalline form of the compound of formula (I) 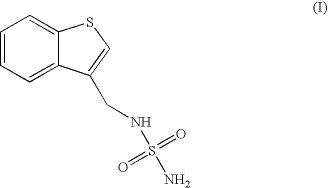
comprising the following X-ray diffraction peaks:

| Position | d-spacing | Relative |
|---|---|---|
| [°2Theta] | [Å] | Intensity [%] |
| 4.44 | 19.91 | 14.21 |
| 5.92 | 14.94 | 100 |
| 13.21 | 6.70 | 10.7 |
| 13.38 | 6.62 | 10.29 |
| 14.80 | 5.99 | 14.5 |
| 16.76 | 5.29 | 54.91 |
| 17.81 | 4.98 | 99.33 |
| 19.00 | 4.67 | 40.12 |
| 21.43 | 4.15 | 11.61 |
| 22.29 | 3.99 | 13.84 |
| 23.82 | 3.74 | 25.39 |
| 24.01 | 3.71 | 14.8 |
| 26.67 | 3.34 | 22.69 |
| 26.95 | 3.31 | 10.39 |
| 36.10 | 2.49 | 11.51 |

2. An anhydrous crystalline form of the compound of formula (I) 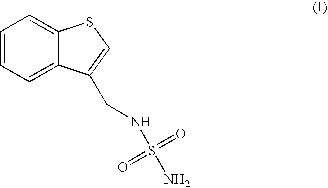
comprising the following X-ray diffraction peaks:

| Position | d-spacing | Relative |
|---|---|---|
| [°2Theta] | [Å] | Intensity [%] |
| 5.84 | 15.14 | 71.34 |
| 13.42 | 6.60 | 42.01 |
| 16.81 | 5.27 | 100 |
| 17.50 | 5.07 | 50.58 |
| 19.30 | 4.60 | 47.27 |
| 20.37 | 4.36 | 15.62 |
| 21.30 | 4.17 | 27.74 |
| 21.81 | 4.07 | 60.53 |
| 22.78 | 3.90 | 33.42 |
| 24.29 | 3.66 | 13.37 |
| 24.99 | 3.56 | 27.14 |
| 26.40 | 3.38 | 21.56 |
| 26.98 | 3.30 | 46.5 |
| 29.89 | 2.99 | 27.38 |

3. An anhydrous crystalline form of the compound of formula (I) 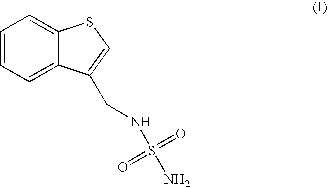
comprising the following X-ray diffraction peaks:

| Position | d-spacing | Relative |
|---|---|---|
| [°2Theta] | [Å] | Intensity [%] |
| 4.46 | 19.80 | 100 |
| 8.90 | 9.94 | 1.61 |
| 13.35 | 6.63 | 18.6 |
| 20.12 | 4.41 | 2.11 |
| 20.61 | 4.31 | 0.51 |
| 21.81 | 4.08 | 0.69 |
| 22.32 | 3.98 | 0.87 |
| 23.44 | 3.80 | 0.80 |
| 24.42 | 3.65 | 0.68 |
| 26.85 | 3.32 | 11.11 |
| 31.43 | 2.85 | 1.1 |
| 33.52 | 2.67 | 0.53 |
| 36.03 | 2.49 | 1.12 |

4. A 1,4-dioxane solvated crystalline form of the compound of formula (I) 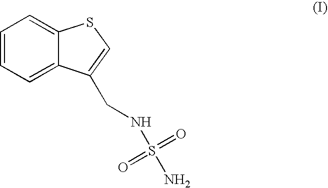
comprising the following X-ray diffraction peaks:

| Position | d-spacing | Relative |
|---|---|---|
| [°2Theta] | [Å] | Intensity [%] |
| 4.44 | 19.89 | 100 |
| 8.86 | 9.98 | 1.67 |
| 13.30 | 6.66 | 26.6 |
| 18.48 | 4.80 | 1.73 |
| 19.38 | 4.58 | 1.99 |
| 19.95 | 4.45 | 2.31 |
| 20.13 | 4.41 | 4.00 |
| 20.60 | 4.31 | 2.34 |
| 21.80 | 4.08 | 4.46 |
| 22.28 | 3.99 | 2.46 |
| 23.43 | 3.80 | 2.71 |
| 24.42 | 3.65 | 5.26 |
| 26.80 | 3.33 | 27.75 |
| 31.38 | 2.85 | 3.07 |
| 33.51 | 2.67 | 1.59 |
| 36.01 | 2.49 | 3.73 |

5. A crystalline form of the compound of formula (I) 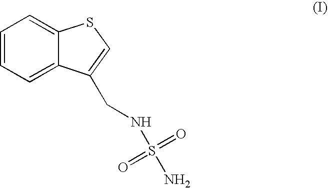
wherein the crystalline form is characterized by a DSC temperature of onset selected from the group consisting of about 95° C., about 98° C. and about 101° C.

6. The crystalline form of claim 5, wherein the crystalline form is characterized by DSC temperature of onset selected from the group consisting of about 95° C.

7. The crystalline form of claim 5, wherein the crystalline form is characterized by DSC temperature of onset selected from the group consisting of about 98° C.

8. The crystalline form of claim 5, wherein the crystalline form is characterized by DSC temperature of onset selected from the group consisting of about 101° C.
